(19) United States
(12) Patent Application Publication
(10) Pub. No.: US 2004/0250305 A1
(43) Pub. Date: Dec. 9, 2004

(54) Adoptive transfer and uses thereof

(76) Inventors: Zuhair A. Latif, Vernon Hills, IL (US); Sean F. Nowlan, Winthrop Harbor, IL (US)

Correspondence Address: Steven F. Weinstock, D-377/AP6A-1, Abbott Laboratories, 100 Abbott Park Road, Abbott Park, IL, 60064-6008, US

(21) Appl. No.: 10/654,723

(22) Filed: Sep. 4, 2003

Related U.S. Application Data
(60) Provisional application No. 60/409,305, filed on Sep. 9, 2002.

(51) Int. Cl. A01K 67/027

(52) U.S. Cl. 800/14; 800/15; 800/17; 800/18

(57) ABSTRACT

The subject invention relates to a method of transferring a specific immune response into a cloned animal. In this manner, one may create a specific, selective, secondary immune response in an otherwise immunologically naïve animal.

The present application claims priority to U.S. Provisional Application Ser. No. 60/409,305, filed on Sep. 9, 2002, hereby incorporated in its entirety by reference.

### BACKGROUND OF THE INVENTION

1. Technical Field

The subject invention relates to a method of transferring a specific immune response into a cloned mammal. In this manner, one may create a specific, selective, secondary immune response in an otherwise immunologically naïve mammal.

2. Background Information

Cloned animals have been utilized for many years in order to produce genetically engineered proteins or factors. In particular, such proteins or factors are expressed in the founder animals and transmitted to the clone. In this manner, one may expand the source of the product of interest as well the supply thereof.

The immune response is a learned and thus adaptive response whereby, following antigenic exposure, cells of the immunized animal undergo a series of stimulation and maturation steps before producing the final product, whether it is a receptor or an immunoglobulin (i.e., antibody) molecule. Therefore, a cloned animal, though genetically predisposed, may or may not necessarily produce the same receptor or antibody specificity upon immunization with the same immunogen, as the founder. Transfer of immune potential from founder to clone, in accordance with the method of the present invention, will substantially increase the opportunity for the expression of those specific immune responses.

Adoptive transfer has been demonstrated for a) identical twins (animals and humans), b) genetically identical individuals of the same species (e.g., highly inbred mice) or, c) genetically close individuals (such as for bone marrow transplants, kidney and other organ donor programs). In the latter case, success is influenced by how close the genetic “match” is (or by how small the “mismatch” is) and by instituting adequate chemotherapy and radiation regiments. However, adoptive transfer, such as that encompassed by the present invention involves quite a different method and has many advantages.

### SUMMARY OF THE INVENTION

The present invention includes a method of transferring an immune response from a founder mammal (e.g., animal) to a cloned mammal (e.g., animal). This method comprises the steps of: a) immunizing a founder mammal with an immunogen; b) cloning the founder mammal; and c) obtaining lymphocytes from the immunized founder mammal and transferring the lymphocytes to the cloned mammal for a time and under conditions sufficient for the mammal to develop the immune response of the founder mammal. The mammal (e.g., animal) may be selected from the group consisting of, for example, a mouse, a rabbit, a sheep, a horse, a pig and a cow. The lymphocytes may be, for example, peripheral blood lymphocytes, lymph node lymphocytes, splenocytes or bone marrow cells. Such lymphocytes may be transferred by transfusion, for example. The immunogen may be any entity capable of eliciting or producing an immune response (e.g., production of antibodies). Examples of suitable immunogens include antigens, epitopes and haptens. The cloning itself is from, for example, somatic cells or embryonic stem cells. Cloning may be achieved by transferring the nucleus from a somatic or embryonic stem cell of the founder animal to an enucleated ovum of a surrogate female, and transferring the resulting blastocyst (or early embryo) into the uterus of the surrogate female during estrous.

### BRIEF DESCRIPTION OF THE FIGURES

FIG. 1 illustrates the method of the present invention in which cells are isolated and purified from the founder animal, the cloned animal is prepared for cellular transfer, and the transfer is performed.

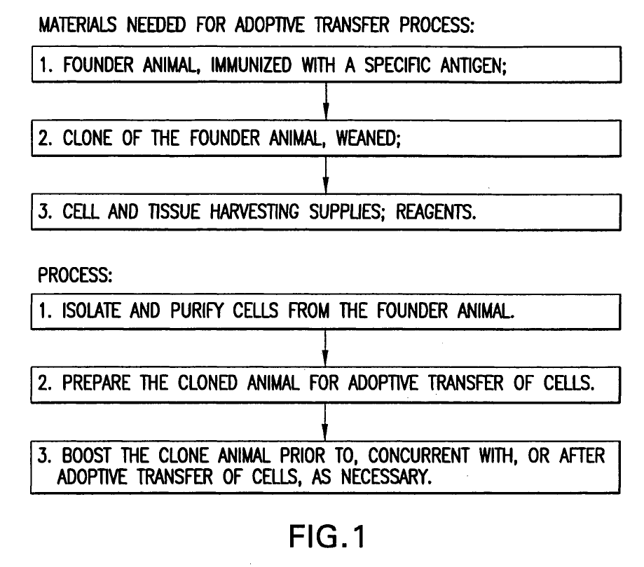

### DETAILED DESCRIPTION OF THE INVENTION

As noted above, a mammal may be cloned; however, the ability of a cloned mammal to make a particular antibody having a particular specificity is a learned response. Furthermore, the cloning process has not been demonstrated to also transfer the immunologic memory from the founder animal to the cloned animal. Therefore, in order to increase the odds in favor of producing a cloned animal with the capability to produce the desired immune response having a defined specificity, a different methodology must be utilized such as that of the present invention.

In particular, the present invention encompasses a method whereby lymphoid cells or lymphocytes (e.g., from whole blood, blood-derived cells, peripheral blood lymphocytes, splenocytes, lymph node lymphocytes or bone marrow cells including stem cells) may be obtained from an animal (i.e., the founder) having a desirable immunological profile (e.g., the demonstrated ability to produce an antibody having a particular specificity). A founder animal is one that is known, following experimentation, to produce a unique immune response that is difficult to duplicate in other animals of the same or different species. Fresh whole blood or cells derived from blood, lymphatic tissue or bone marrow are then suspended in freeze media containing nutrients (e.g., fetal calf serum) and, for example, DMSO (dimethyl sulfoxide) as a cryoprotectant and stored frozen in, for example, liquid nitrogen. Once a cloned animal is available (created by using the founder animal), it may then be injected with fresh or preserved cells from the founder animal. Since the transfused cells are genetically identical to the clonal host or founder animal, they should not invoke immune rejection and are expected to successfully repopulate the lymphoid organs in the host. As such cells contain immunologically competent memory cells, the stimulation thereof in the cloned animal, by in vivo challenge, will produce the desired anamestic immune response of the founder animals.

The need for the present invention is significant. Such a need may be, for example, illustrated as follows:

An essential and critical component of a diagnostic assay for T4 is sheep anti-T4 serum that is immobilized onto a solid phase (e.g., microparticles). In combination with a conjugate made up of T3 (Triiodothyronine, an analog of T4) and alkaline phosphatase, the sheep serum confers basic critical quality attributes required to generate a distinct standard calibration curve and allow for an estimate of FT4 in patient samples.

The serum is developed by immunizing sheep with T4-Tg complex. Thyroxin (T4) is coupled onto a protein carrier molecule (porcine thryoglobulin or Tg), then emulsified in an adjuvant prior to injection into sheep. This is a classical approach to raising needed immune responses in experimental animals. Historically, however, this method of immunization produced antibodies recognizing T4 molecules; yet, in the great majority of instances, the resulting sera does not perform adequately in diagnostic tests.

Success of adoptive transfer requires that the source and the destination animals either be genetically compatible (as in identical twins, clones, highly inbred species as is the case in some mice) or the recipient animal (destination) be immunologically suppressed through the use of chemical agents and radiation.

It is not readily understood if such a rare and unique immune response is dictated solely by the animal's genetic background or to what degree the response is confounded by a variety of presently unknown factors. On the basis of theory alone, however, a large contributor to the uniqueness of such a response is the genetic make up of these responders. The low efficiency and unpredictable response is an obstacle to providing long-term resources and reagent safety stock and therefore jeopardizes the availability of test material. However, if an immunologic responder animal is cloned, in accordance with the present invention, the probability of raising a clone with immunologic potential similar to that of the founder animal is significantly enhanced. Moreover, the adoptive transfer of immunologically competent lymphoid cells from the founder to the clone will further enhance the opportunity of duplicating the immune competency of the founder animal without the risk of immune rejection.

In view of the above, one purpose of the present invention is to produce a cloned animal with the same immune capacity and immunological specificity, as the founder animal with respect to a specific antigen. The transfusion may be preceded by, followed by or concurrent with immunization and/or boosting by an immunogen that has been demonstrated to illicit a particular immune response to yield the desired antibody specificity. Other manipulations may also be attempted to increase the likelihood of producing the needed antibody depending on the success of this transfusion approach. For instance, one possible manipulation is to boost a sheep which has previously been immunized using T4-Tg immunogen, with T4 coupled to a different carrier molecule such as KLH (Keyhole limpet hemocyanin).

The antibodies produced by the cloned animal may be used for many purposes. For example, the antibodies may be utilized in diagnostic assays as well as for therapeutic purposes. The present invention therefore will allow for the production of an endless supply of such antibodies without the concern of maintaining the desired immunological response of the founder animal.

The present invention may be illustrated by the use of the following non-limiting examples:

### EXAMPLE I

### Adoptive Transfer of Immunity to a Cloned Animal

Initially, fucosyl transferase transgenic mice (or a group of animals of the same species) are immunized with an antigen such as T4-TG. The immunized mice are then cloned using fibroblast cells as nuclear donors. At adulthood, the cloned mice are then divided into two groups. Immune splenocytes from the immunized founder mice are then obtained and transferred to the Group I mice (Adoptive Transfer Group). In contrast, naïve splenocytes are obtained from un-immunized mice and transferred to Group II (Negative Control Group). Both groups of cloned mice are challenged with T4-TG antigen. The antibody response or titer produced against the T4 hapten is measured in both groups and compared.

If adoptive transfer is successful, Group I mice (animals transfused with immunologically trained cells) show a secondary immune response (high titer, specific antibody) while Group II mice (animals transfused with immunologically naïve cells) show only a primary immune response (low titer and less specific antibody), such as in vaccination. Primary and secondary immune responses are better understood in the context of commonly used vaccines. A vaccine is designed to train the immunologically naïve cells to become “educated” immune cells. Once immune (or educated) cells encounter a real infection, they respond more rigorously (e.g., higher antibody level, i.e., higher titer) and more specifically than an otherwise uneducated or naïve cell.

1. A method of transferring an immune response from a founder mammal to a cloned mammal comprising the steps of:
a) immunizing a founder mammal with an immunogen;
b) cloning said founder mammal;
c) obtaining lymphocytes from said immunized founder mammal and transferring said lymphocytes to said cloned mammal for a time and under conditions sufficient for said cloned mammal to develop said immune response of said founder mammal.

2. The method of claim 1 wherein said mammal is selected from the group consisting of a mouse, a rabbit, a sheep, a horse, a pig and a cow.

3. The method of claim 1 wherein said transfer of lymphocytes is by transfusion.

4. The method of claim 3 wherein said lymphocytes are selected from the group consisting of peripheral blood lymphocytes, lymph node lymphocytes, splenocytes and bone marrow cells.

5. The method of claim 4 wherein said lymphocytes are splenocytes.

6. The method of claim 1 wherein said immunogen is selected from the group consisting of an antigen, an epitope, a hapten, and a portion thereof.

7. The method of claim 1 wherein said cloning is from somatic cells or embryonic stem cells.

8. The method of claim 7 wherein cloning is achieved by transferring the nucleus from said somatic or embryonic stem cell of said founder animal to an enucleated ovum of a surrogate female, and transferring said resulting blastocyst into the uterus of said surrogate female during estrous.
